(19) United States
(12) Patent Application Publication
(10) Pub. No.: US 2006/0135907 A1
(43) Pub. Date: Jun. 22, 2006

(54) Device and method for checking a medical device

(76) Inventors: Axel Remde, Luetzelflueh (CH); Gilbert Schiltges, Kirchberg (CH)

Correspondence Address: DORSEY & WHITNEY LLP;INTELLECTUAL PROPERTY DEPARTMENT, 50 SOUTH SIXTH STREET, MINNEAPOLIS, MN, 55402-1498, US

(21) Appl. No.: 11/304,505

(22) Filed: Dec. 15, 2005

Related U.S. Application Data
(63) Continuation of application No. PCT/EP04/05037, filed on May 11, 2004.

(30) Foreign Application Priority Data
Jun. 17, 2003 (DE) 103 27 261.5

(51) Int. Cl. A61M 31/00

(52) U.S. Cl. 604/67

(57) ABSTRACT

A device integrated with and/or operably coupled to a medical device, including an acoustic element for detecting a sound associated with the medical device to monitor and/or assess the performance and/or condition of the medical device. The invention encompasses a method for assessing the operation, performance and/or condition of a medical device wherein a sound associated with the medical device is detected and analyzed.

### CROSS-REFERENCE TO RELATED APPLICATION(S)

This application is continuation of International Patent Application No. PCT/EP2004/005037, filed on May 11, 2004, which claims priority to German Application No. DE 103 27 261.5, filed Jun. 17, 2003, the contents of both applications are incorporated in their entirety by reference herein.

### BACKGROUND

The present invention relates to medical devices, systems and assemblies, and methods of making and using such devices, systems and assemblies. More specifically, the present invention relates to monitoring, diagnosing and/or assessing the performance, operability and/or operational state of medical devices, including non-implantable devices.

Exemplary medical devices include injection devices, devices for measuring the concentration of a substance, infusion pumps, and devices for determining a medical function or condition, for performing drug or substance delivery, for conducting measurements, etc. In accordance with the present invention, the operable condition and/or faults or performance anomalies that may be associated with such devices may be detected. Examples of such faults or operating conditions include catheter or cannula blockage, an occlusion, a leak, a damaged or worn drive mechanism, component misalignment, a gear mechanism in an infusion pump that has been damaged by wear or external impact, etc. The assessment or detection may be performed by operational diagnostic functions of the device, or may be performed by an integrated or operably coupled checking device.

Infusion pumps, for example, can be used outside the body and serve for providing a dosed supply of substances, such as insulin or hormones, to a body. In this case, the correct functioning of such an extracorporeal infusion pump should be monitored to ensure correct administration of medicaments. And, in the case of a detected fault, a warning should be produced. If appropriate, further suitable measures, such as interrupting or halting operations could be performed. In the case of infusion pumps commonly in use, the mechanism provided for the dosed administration of a substance may not be not directly accessible for defect detection.

There are known infusion pumps in which catheter or needle blockages are detected by means of measuring a reaction force associated with the drive or gear mechanism or by means of measuring the current required by the motor. However, measuring the reaction force of the gear mechanism requires complex equipment and is expensive, and may adversely influence performance parameters of the infusion pump, such as the rigidity of individual components and the overall size of the infusion pump. The detection of a malfunction of the infusion pump by means of measuring the motor current has a slow response, as a result of which malfunctions may only be established relatively late. In general; occlusions and other faults that may impair the dosed administration of a substance or medicament may only be detected imprecisely or too late by the aforementioned methods, or may not be detected at all.

U.S. Pat. No. 4,985,015 discloses an implantable dosing device in which an armature firmly connected to a piston is arranged in such a way that an annular surface of the armature lies opposite an annular surface of a cylinder housing, so that a noise that can be distinguished from a normal pumping noise is produced when these two surfaces hit each other. This stopping noise is used for controlling and monitoring the piston pump.

European Patent EP 0 519 765 B1 discloses an implantable infusion pump, an electronic stethoscope being placed onto the skin over the implanted infusion pump and an acoustic signal being measured when the pump mechanism is in operation.

### SUMMARY

It is an object of the present invention to provide a device and method for checking or monitoring the condition and/or performance a medical device.

In one embodiment, the present invention comprises a device integrated with and/or operably coupled to a medical device, including an acoustic element for detecting a sound associated with the medical device to monitor and/or assess the performance and/or condition of the medical device. The invention encompasses a method for assessing the operation, performance and/or condition of a medical device wherein a sound associated with the medical device is detected and analyzed.

In one embodiment, the present invention comprises an acoustic element integrated with and/or operably coupled to a medical device for detecting a sound associated with the medical device to monitor and/or assess the performance and/or condition of the medical device. A method for assessing the operation, performance and/or condition of the medical device wherein a sound associated with the medical device is detected and analyzed is encompassed.

In one preferred embodiment of the present invention, an extracorporeal infusion pump is involved. However, other medical devices may be checked using methods and systems according to the present invention, including injection devices, delivery or administering devices, injection pens, measuring devices, etc.

The device for checking a medical device, according to one embodiment of the invention, is preferably used outside the body. For example, an extracorporeal, non-implanted infusion pump may include an acoustic transducer for recording sounds emitted by the device during operation. Sounds, which may be structure-borne sounds or airborne sounds, can be detected by one or more acoustic transducers or measuring transducers based on various physical principles. For example, electrodynamically, capacitively, piezoelectrically or piezoresistively operating transducers may be used in accordance with embodiments of the present invention to detect sounds. The sound detected by an acoustic transducer from the drive system of an extracorporeal infusion pump of the syringe pump type, for example, can be evaluated in an evaluation unit, which detects the state, the operating behavior, or, generally, the system behavior or performance of the medical device. Thus, the performance of the medical device, and sensed faults in the medical device and/or in the functioning of the medical device can be detected and/or assessed. Methods and systems of the present invention use characteristics, including, e.g., intensity of a sound that is associated with and/or emitted by a medical device, such as by a drive system of a pump, to determine the operating state of the medical device because sound characteristics are influenced by the state and the operating situation of the device.

In some embodiments, acoustic transducers may be coupled to the medical device by attachment to or integration with the medical device. Using the acoustic transducers, sounds transmitted through the body of the medical device may be sensed and/or measured. Integrated transducers may measure device sounds more closely since structure-borne sound measurement is less sensitive to environmental influences, such as interfering ambient noise.

In general, however, it is also possible to detect the sounds emitted by an extracorporeal infusion pump by an acoustic transducer which is not physically connected to the medical device and is at a certain distance from it, although, in some embodiments, preferably only air should lie between the medical device and the acoustic transducer.

According to one preferred embodiment of the present invention, a vibration device produces a known oscillation or vibration pattern, which is transmitted to the medical device and may be detected by an acoustic transducer provided in or on the medical device. For example, an external oscillation or vibration device can transmit oscillations to the device. On the basis of the structure-borne or airborne sound emitted by the medical device and detected by an acoustic transducer, it can be determined whether the oscillations produced by the vibration device or the sound produced is propagated in a way that is expected in the case of an intact and correctly functioning medical device. If a different or anomalous vibration or sound pattern occurs and is detected, it may be concluded that there is a defect or a fault in the operation of the device. If, for example, sound measurements of oscillations or sound patterns of medical devices that have a defect, such as a crack in the housing, or a malfunction of the drive are known and stored in a database, it is possible to determine from the measured sound which malfunction or which defect is present in or on the medical device. In one embodiment, a functional check of a vibration alarm may also be performed.

In one embodiment of the present invention, a signal output device is provided on a medical device that outputs optical and/or acoustic signals upon detection of a fault or operating state of the medical device. In one example, a first signal is output in the form of a green LED if it is established by an acoustic transducer and a downstream evaluation unit that the medical device is intact and functioning correctly. A second signal may be output by a yellow LED, if it is detected that there are deviations from a prescribed sound pattern, and consequently there is possibly a defect or faulty operating state. A third signal may be output by a red LED, if it is determined that a fault has occurred. In addition to or alternative to signalling the status of the medical device optically, audible signals may be output. In another example, a vibration device may be associated with the medical device and may be activated to indicate to a user that an action requested by the user is not correctly performed or that the device has developed a fault.

According to embodiments of the present invention, various faults or faulty operating states may be detected using a checking device that carries out a sound measurement of a medical device. Faults that may be detected include catheter blockages, occlusions, or bubbles, worn or soiled threaded rods, which serve in the case of infusion pumps for the dosed delivery of a substance, inadequate or absent lubrication, drive faults, such as knocking bearings or tooth breakage, etc. In accordance with another embodiment of the present invention, device checking may serve a preventative function and may detect and monitor the faultless or normal functioning of a medical device, such as an infusion pump. For example, faultless monitoring may include the monitoring of the delivery of a substance contained in a pump, ampoule or external supply, checking of an alarm device, such as a vibration device, assessment of the abrasion or, generally, the wear, of the medical device, or the detection of an impact, which is usually also accompanied by the emission of sound.

According to a further aspect of the present invention, a diagnosis, performance assessing or diagnostic station (e.g., a “docking” type visit) for a medical device may be provided. The diagnostic station may be provided in order to detect defects or malfunctions related to, in or on the medical device, and/or in order to determine that the medical device is mechanically and structurally sound, and/or is performing properly. According to one embodiment, the diagnostic station may include a recording or coupling device coupleable to the medical device by direct contact, or, for example, by electromagnetic waves, such as radio, infrared radiation or capacitive or inductive coupling. According to the invention, the diagnostic station may include an evaluation unit for evaluating sound signals recorded by an acoustic transducer at the medical device, or at the diagnostic station.

In some embodiments memory may be provided or operably coupled to a medical device and/or diagnostic station for storing and/or processing sound pattern or other characteristics of medical device. The “remembered” characteristics may correspond to a faultless state of operation, and, optionally, fault states or defects associated with the medical device. This enables the diagnostic station to compare detected sound signals with stored sound signals to determine whether a medical device has defects, is malfunctioning, or is functioning correctly. In accordance with a further embodiment, the specific defect or malfunction may be identified based on the signal comparison.

In one embodiment of the present invention, a diagnostic station with an acoustic recording device may detect a sound emitted by a medical device and communicate it to an evaluation unit. An acoustic transducer of the diagnostic station may be configured to detect medical device sounds transported through air which may be recorded by the recording device. In addition, the transducer may be adapted to be attached temporarily or permanently to the medical device. This may allow sound transported through the body of the medical device to be detected, while minimizing the detection of interfering ambient noises, which would enhance accurate detection and evaluation of the detected sound signals.

According to a further aspect of the present invention, a method for checking a medical device may include analyzing detected sounds or oscillations emitted by the device. In some preferred embodiments, a sound emitted by the device is detected directly, so that the sound at the device itself is detected by an attached acoustic transducer, thereby minimizing the amount of ambient noise recorded. The evaluation of the detected sound or sound signal may take place automatically, for example by a computer-aided system, or by an expert who is familiar with the sound patterns or sound signals emitted by properly functioning and malfunctioning medical devices.

According to an embodiment of the present invention, the sound detection for checking the medical device is preferably carried out continuously or virtually continuously, for example, with each functional operation or discharge, in order to constantly monitor the medical device, such as an infusion pump, and to immediately detect occurring faults or malfunctions.

According to another embodiment of the present invention, the detection of sound may also be carried out temporarily, periodically, or between prescribed sound measurement time intervals. Furthermore, it may also possible for the sound measurement and medical device checking to be carried out upon a user command, or automatically upon detection of a trigger or actuation. For example, a trigger may be a specific event, such as an impact or a drug interaction.

According to certain embodiments of the present invention, impact detection may be carried out. An impact often produces a specific characteristic sound signal, which may be detected by a transducer. Once an impact been detected by means of receiving a sound signal indicative of an impact, a functional check of the medical device may optionally be carried out. For example, the drive system and/or a vibration device present in the medical device may be activated, resulting in the production of oscillations which propagate through parts of the device or the entire medical device. Oscillations may be detected in order to check from the detected sound pattern whether or not the impact resulted in any damage or malfunction of the device, e.g., in the drive system, casing, etc.

In accordance with another embodiment of the present invention, the medical device may output a warning signal and/or be blocked or shut down completely if a malfunction or fault is detected.

In some preferred embodiments, detected sound signals or derived variables, such as frequency spectra, are stored to have a recording of the operation and possible disturbing influences, such as impact or malfunctions, of a medical device. This enables the recorded signals to be evaluated to check the functional capability and operational reliability of the medical device. Storage of the recordings may be in the medical device, and/or in an external storage device. In one example, data may be transmitted to an external storage device over a line or a wireless connection, such as by radio or infrared signals.

### BRIEF DESCRIPTION OF THE DRAWINGS

FIG. 1 shows a schematic view of a non-implantable infusion pump to be used outside the body;

FIG. 2 is a circuit diagram of one embodiment of a device according to the invention;

FIG. 3 is a circuit diagram of another embodiment of a device according to the invention;

FIG. 4 is a circuit diagram of another embodiment of a device according to the invention;

FIG. 5, including FIGS. 5a and 5b, depicts a signal of a vibration device of an infusion pump recorded by an airborne-sound acoustic transducer in the time and frequency ranges;

FIG. 6 depicts the effective power of a sound signal as a function of the delivered amount of insulin in the case of an occlusion;

FIG. 7, including FIGS. 7a and 7b, depicts the running noises recorded in the case of a faultlessly operating infusion pump in the time range and the corresponding power spectrum in a characteristic frequency range; and

FIG. 8, including FIGS. 8a and 8b, depicts signals produced in the case of a tooth breakage in the gear mechanism of an infusion pump.

### DETAILED DESCRIPTION OF THE DRAWINGS

With regard to fastening, mounting, attaching or connecting the components of devices of the present invention, unless specifically described as otherwise, conventional fasteners such as screws, rivets, toggles, pins and the like may be used. Other fastening or attachment means appropriate for connecting components include friction fitting, adhesives, welding and soldering, the latter particularly with regard to electrical or processing components or systems of the devices. Any suitable electronic, electrical, communication, computer or processing components may be used, including any suitable electrical components and circuitry, wires, wireless components, sensors, chips, boards, micro-processing or control system components, software, firmware, hardware, etc.

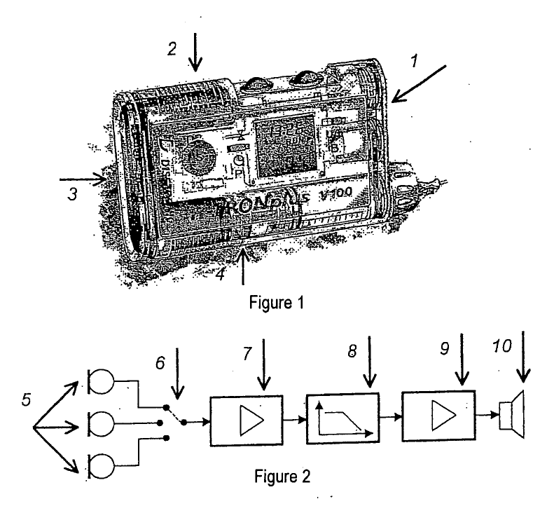

FIG. 1 shows a non-implantable infusion pump 1 to be used outside the body, whereby insulin or other substance may be administered in a dosed manner, in accordance with an embodiment of the present invention.

The insulin pump 1 may be of the syringe pump type and preferably has suitable placement points for the placement of acoustic transducers, motor 2, gear casing 3 and clam nut 4. Insulin pump 1 is not limited to a syringe-type pump, but rather may be of any suitable type of pump or medical device. According to one embodiment, acoustic transducers may be permanently attached or integrated to pump 1. Alternatively, transducers may be releasably attached to or integrated with pump 1, for example by suction cups or adhesive wax.

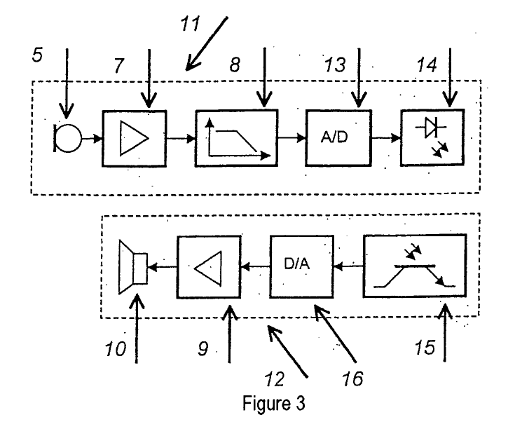

FIG. 2 shows a measuring arrangement with a number of measuring transducers 5, a signal changeover switch 6, a preamplifier 7, a filter 8, a reproducing amplifier 9, and a playback device 10, such as headphones or a loudspeaker. It is possible for any number or type of amplifiers, filters, noise suppression systems, or other suitable electronic components, analog or digital, to be used, in order to prepare the sound signal detected by a measuring transducer or transducers 5 or carry out preprocessing. The sound signal detected by measuring transducer 5 attached to insulin pump 1 is output via a playback device 10 and may be evaluated by an expert, who assesses the signal on the basis of his expertise and experience with regard to anything possibly anomalous or deviational from a desired signal. Consequently, an insulin pump can be checked after suffering an impact, or the state of wear can be assessed.

As an alternative to the embodiment shown, it is also possible to provide a single or a number of measuring transducers 5 in the case of a diagnostic station, into which the pump is placed or clamped, the components shown in FIG. 2 also being able to be integrated in a diagnostic station. Optionally, a number of measuring devices and auxiliary means for detecting the state of the medical device may be integrated or connected, such as in a storage device for documentation, or by an oscilloscope for the graphic representation of the sound signals in the time or frequency range.

As an alternative or in addition to the evaluation of the detected sound signals by an expert, measuring signals may also be fed in to an evaluation unit, where sound signals detected by the measuring transducer or transducers 5 may be digitized and transmitted to a computer or processing system for further processing, evaluation, analysis, classification and/or storage by software.

FIG. 3 shows a second embodiment of a device according to the present invention that includes one or more acoustic transducers 5 being integrated in an infusion pump 1, or attached to it. Signals detected from the transducers for determining medical device status may be communicated and/or read out via interface 14. Analog sound signals recorded by measuring transducer 5 in the interior 11 of pump 1 may be digitized by an A/D converter 13 via amplifier 7, and, optionally, via filter 8. Signals may be transmitted via interface 14, to an evaluation unit 12, where the signal may be converted into an analog signal in D/A converter 16 and fed via reproducing amplifier 9 to playback device 10 for evaluation. In one example, interface 14 may be a serial IR interface present in the infusion pump. It may also be possible, however, to design the interface as a radio, capacitive, inductive, cable or other suitable interface. Optionally, the transmission may be carried out in an analog form, thereby removing the requirement of A/D and D/A converters. Similarly, it may be possible to carry out filtration, processing, or preparation of the sound signals detected by measuring transducer 5 in an evaluation device 12. This may be carried out in addition to signal processing in the interior 11 of infusion pump 1, or without the prior signal processing or preparation in pump 1, thereby transmitting only the directly detected sound signals from pump 1 to evaluation unit 12. The recording and/or output of the sound signal may take place continuously or by means of a pump control system, which, for example, may receive a signal from a user, or carry out a functional check after detected impact or routinely or periodically.

If the measured-value or acoustic transducer 5 is integrated directly in the pump, it can be precisely placed directly at a sound source and directly detect a sound signal emitted by a specific functional group, largely avoiding attenuation and undefined filtering of the sound signal to be detected, for example by the housing of the infusion pump 1.

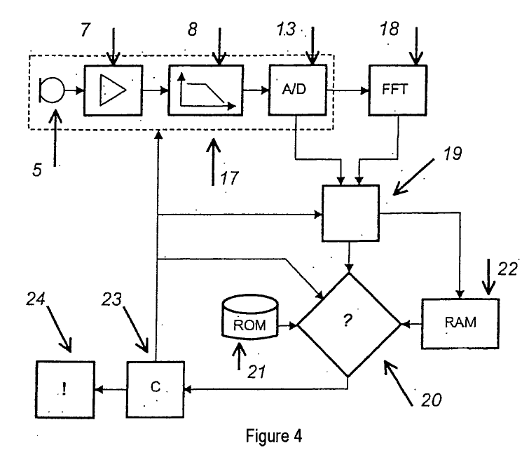

FIG. 4 depicts a circuit diagram of an embodiment of the invention, a measuring arrangement 17 having a measuring transducer 5, an amplifier 7, a filter 8 and an A/D converter 13. The sound signal detected by the measuring arrangement 17 is transformed from the time range into the frequency range by a fast Fourier transformation (FFT) device 18. In the signal processing element 19, the signal may be further processed in the time and/or frequency range, for example, a digital filtration may be carried out. The power spectrum may be calculated, and/or variables that are characteristic of the checking of the infusion pump, such as peak values or effective values, may be assessed. The analysis element 20 compares the signals and characteristic variables calculated or evaluated by the signal processing element 19 with comparison and reference data, which are stored, for example, in a read-only memory (ROM) 21, or which have been calculated in prior measurements and stored as adaptive reference values in a random-access memory (RAM) 22. The memories ROM 21 and/or RAM 22 may be integrated in the pump I and/or arranged in an external analysis and evaluation unit.

The analysis element 20 carries out the evaluation of the current system state, e.g., it is established whether operation is normal, or whether there is a fault state or which fault state or which operating malfunction is occurring. The result of the analysis carried out by the analysis element 20 is transmitted to the control system 23 of the pump, which in the case of a fault instigates, for example, the output of an alarm signal via a user interface 24, such as a display, a buzzer or a vibration device, and in the case of acute faults, can instigate further measures, such as the shutting down of the pump 1.

As is true of the previously described exemplary embodiments, the embodiment of the invention shown in FIG. 4 can operate both continuously and non-continuously and can be activated by the pump control system 22 as and when required. Optionally, it is possible for individual components of the circuit shown in FIG. 4 to be parameterized in a suitable manner.

It is generally the case with all embodiments that the extraction of features from detected sound signals may take place by suitable circuits entirely or partly with an analog or digital signal, for example by using filters, peak-value rectifiers, mean-value rectifiers or other suitable devices. Furthermore, it is possible only to take into consideration in the pump those fault situations that require a direct reaction, such as occlusions or a defect of an alarm device. Further functions for general diagnostic purposes may be carried out outside the pump 1 in a diagnostic station. In this instance, signals are made available by a measured-value transducer 5 arranged in the pump and transmitted to the outside via an interface, as shown in FIG. 3.

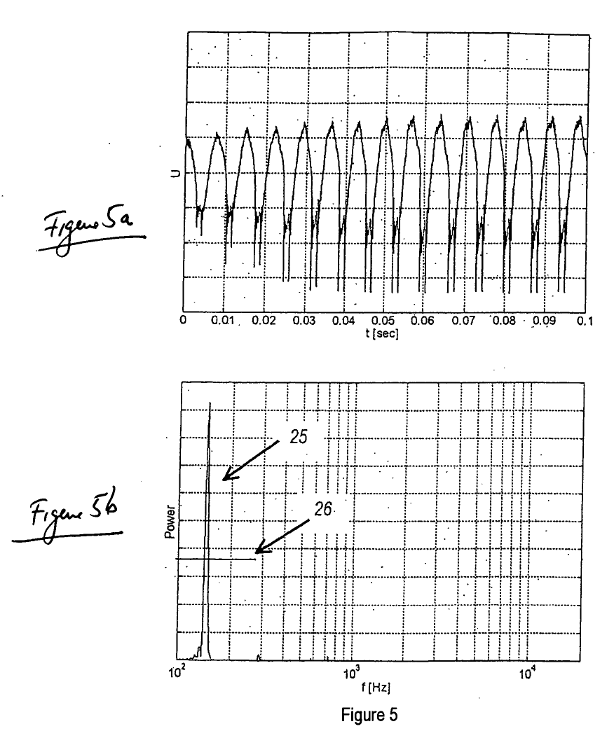

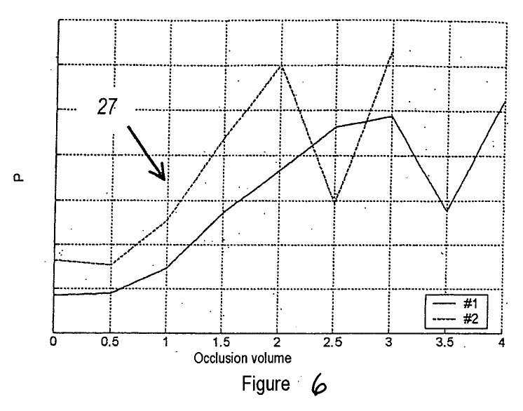

FIG. 5a shows the signal of a vibration alarm 24 associated with an insulin pump 1, recorded outside infusion pump 1 at motor 2 in by an airborne-sound acoustic transducer, and FIG. 5b shows the associated power spectrum in the frequency range of 100 Hz to 20 kHz. Since the vibration frequency of f≅140 Hz is known and approximately constant, an automatic functional check can be performed by filtration with a narrowband bandpass filter of the center frequency approximately in the range of the vibration frequency. A subsequent comparison with a threshold value stored in a read-only memory 21 may be performed. As a result, it may be determined whether a vibration alarm device is operating satisfactorily or whether infusion pump 1 has a fault.

As an alternative, the power in the transmission band of the bandpass filter can be considered absolutely and in relation to the overall power of the sound signal, whereby it is possible to check infusion pump 1 or a vibration alarm device for faults.

With the same method, an acoustic alarm transmitter can also be checked. This check may take place either with every self-test of the pump, for example after exchanging or loading a medicament ampoule, or when an activation is effected by the pump control system 23.

FIG. 6 illustrates the effective power of a recorded sound signal, resulting from a running noise of a drive, this power being equivalent to the square of the effective value of the signal voltage, as a function of the delivered amount of insulin or the occlusion volume in the case of an occlusion, in two examples. As illustrated in FIG. 6, the effective value increases in the region marked by the arrow 27 after the occurrence of the occlusion, whereby an occlusion may be detected.

A distinction may be drawn between two operating states of an infusion pump. In the case of (virtually) continuous delivery of relatively large amounts of medicament, in particular in the case of bolus deliveries, with a correspondingly long motor running time, usually in the range of a few seconds, measurements of the effective sound power are carried out over the entire running time of the motor and stored in the memory 22. It is assumed that an occlusion occurs if, in the case of the individual measured values of the effective sound power, a significant rising trend is exhibited, as shown by way of example in FIG. 6. For the detection of the trend, various methods can be used, for example, in the case of one variant, an alarm being triggered if the individual measured values of the effective sound power respectively rise by more than a prescribable minimum or acceptable value.

In addition or as an alternative, the exceeding of a limit value of the effective sound power on one or more occasions can be checked, this limit value either being stored in the read-only memory 21 of the pump 1, or stored as an adaptive variable in the memory 22. In this case, the fixing of the limit value for the power may take place, for example, on the basis of the sound measurement in the case of the first discharge (priming) after the use of a new medicament ampoule.

In the case of a series of small medicament deliveries, in particular basal deliveries, with correspondingly short motor running times, analogous methods can be used, for example the assessment of a sequence of successive discharges may be used as measured diagnostic values.

As an alternative or in addition to the determination of the absolute sound power, an analysis of the spectral composition may be carried out on the basis of a Fourier transformation by a FFT element 18. For example, an increase of the high frequency components in the amplitude or power spectrum is characteristic of an occlusion and can be detected by an expert or by suitable software. In this case, the amplitude or frequency spectrum may also be compared with one or more reference spectra, for example to detect the occurrence of the occlusion, but also to make a more detailed statement concerning the occlusion occurring or to detect other fault states.

If, for example, defects or contaminations of the drive system are to be detected, the detected sound signal can be investigated for fluctuations of the noise level. Contaminations, in particular due to the penetration of foreign particles into the drive system, bring about both an increase in the noise level, as manifested by the effective value of the sound signal recorded, and a strong fluctuation of this noise level, on account of the increased friction. The rise of this noise level can be compared to the effective value of the recorded sound signal with a prescribed limit value. As already mentioned above, this limit value may be fixed or adaptively chosen. The range of fluctuation of the sound emission may be determined by a statistical analysis of the effective value or by any other suitable characteristic variable, such as a peak value of the sound signal or power spectrum.

As described above, depending on the discharge amount of the infusion pump, the analysis of the sound signal can use as measured values either a number of measurements carried out during one discharge or, a number of successive discharges. Similarly, an analysis of the fluctuations of the noise level in the frequency range is possible.

Defects in the drive system, such as in the motor and/or in the gear mechanism, can have similar effects on the running noise of the infusion pump as contaminations. Such defects, for example in the case of tooth breakages, are often characterized by impulse noises, the frequency of which corresponds to the rotational speed of the respective gear stage.

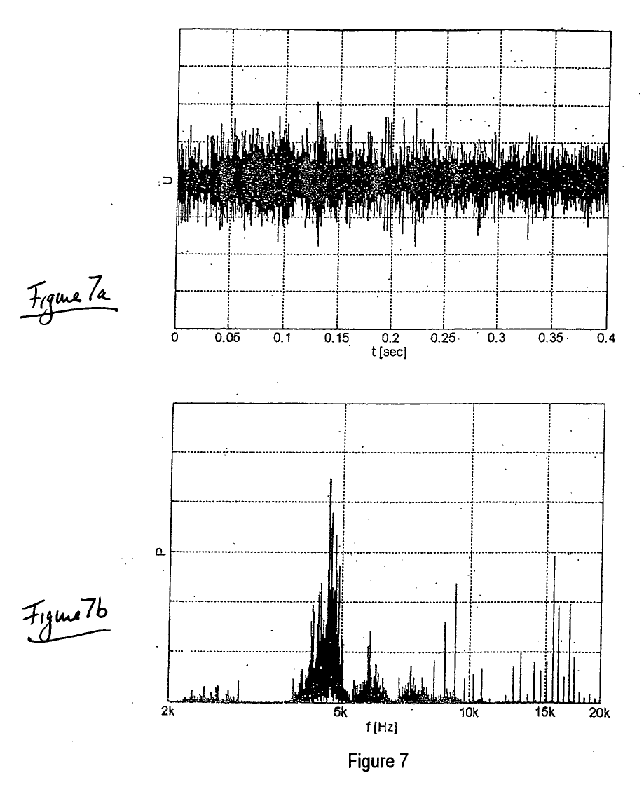

FIG. 7a shows the running noises recorded with a faultless pump in the time range and, and FIG. 7b shows the associated power spectrum in the characteristic frequency range of 2 kHz to 20 kHz.

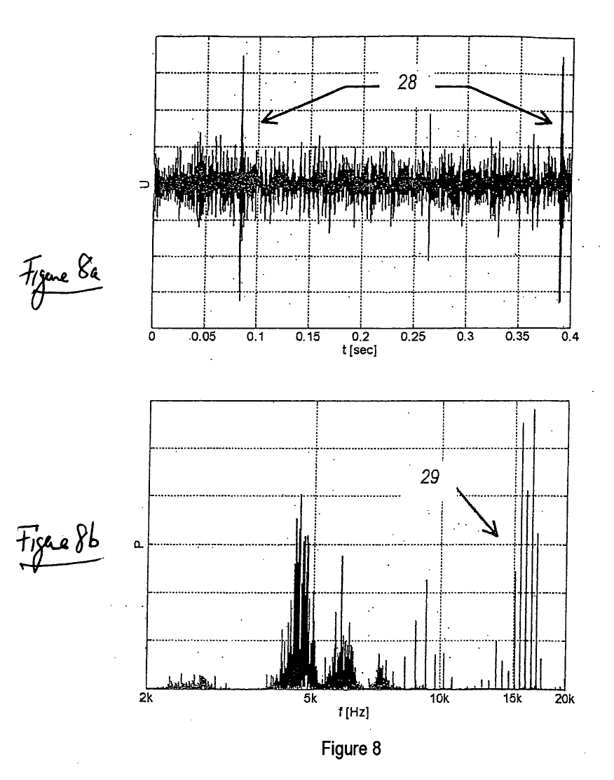

FIGS. 8a and 8b depict the same variables as FIGS. 7a and 7b, but with a tooth breakage in the gear mechanism of the infusion pump. In this case, the acoustic transducer was arranged in the casing 3 in FIG. 1. In the time signal, the pulses 28 respectively occurring when the defective tooth engages are clearly visible. In the frequency spectrum, these pulses bring about a clear increase in power in the upper frequency range of 10 kHz to approximately 20 kHz, as represented by the arrow 29. The detection of such pulses may take place, for example, by a high-pass filtering in the time or frequency range with a subsequent threshold value comparison.

Embodiments of the present invention, including preferred embodiments, have been presented for the purpose of illustration and description. They are not intended to be exhaustive or to limit the invention to the precise forms or steps disclosed. The embodiments were chosen and described to provide the best illustration of the invention and its practical application, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

What is claimed is:

1. A device for checking a medical device, comprising at least one acoustic transducer, which can detect a sound emitted by the medical device in order to check the medical device for faults.

2. The device as claimed in claim 1, wherein the medical device is an infusion pump for extracorporeal use.

3. The device as claimed in claim 1, further comprising an evaluation unit for receiving signals from the at least one acoustic transducer for detecting the occurrence of a fault.

4. The device as claimed in claim 1, wherein the at least one acoustic transducer is one of permanently connected to or integrated in the medical device.

5. The device as claimed in claim 1, wherein the at least one acoustic transducers is configured to record at least one of airborne sound and structure-borne sound.

6. The device as claimed in claim 1, wherein said medical device includes the device for checking the medical device.

7. The device as claimed in claim 1, further comprising a signal output device for the output of at least one of optical, acoustic and movement signals.

8. The device as claimed in claim 1, wherein said at least one transducer detects sound emitted continuously or intermittently.

9. The device as claimed in claim 1, wherein said device for checking detects an impact on the medical device based on the analysis of the sound emitted.

10. The device as claimed in claim 1, further comprising an alarm for outputting a warning signal or discontinuing the operation of the medical device based on at least one of a detected fault the detected functioning of the medical device, said fault and functioning detected by means for analyzing the detected sound.

11. The device as claimed in claim 1, further comprising a storage device for storing at least one of sound detected by the device and the result of an analysis of the sound.

12. The device as claimed in claim 1, wherein said acoustic transducer is configured to detect at least one of an oscillation, vibration, or impact produced in or on the medical device.

13. A device for checking a non-implantable medical device comprising:
one or more acoustic transducers for detecting emitted sounds from said non-implantable medical device, wherein at least one of said transducers detects a sound related to a structural or mechanical characteristic of said non-implantable medical device;
a storage device for storing signals produced by said one or more acoustic transducers; and
an output device for outputting signals stored on said storage device.

14. The device as claimed in claim 13, further comprising an analysis device operably coupled to said one or more acoustic transducers for analyzing signals produced by said one or more acoustic transducers.

15. The device as claimed in claim 14, further comprising an alarm operably coupled to said analysis device, said alarm triggered based on said signal analysis.

16. The device as claimed in claim 13, wherein the non-implantable medical device comprises a non-implantable infusion pump.

17. The device as claimed in claim 13, wherein a second transducer of the one or more acoustic transducers is configured to detect air bubbles in said infusion pump.

18. The device as claimed in claim 13, wherein said sound related to the structural or mechanical characteristic comprises a sound related to a characteristic indicative of structural stability.

19. The device as claimed in claim 13, wherein said sound related to the structural or mechanical characteristic comprises a sound indicative of functional capability or operational reliability.

20. The device as claimed in claim 13, wherein said sound related to the structural or mechanical characteristic comprises a sound indicative of a structural or mechanical defect.

21. The device as claimed in claim 13, wherein said sound related to the structural or mechanical characteristic comprises a sound indicative a mechanical gear breakage.

22. The device as claimed in claim 13, wherein said sound related to the structural or mechanical characteristic comprises a sound indicative of the need for structural or mechanical maintenance.

23. The device as claimed in claim 13, wherein said sound related to the structural or mechanical characteristic comprises a sound indicative of an impact.

24. The device as claimed in claim 13, wherein at least one of the one or more acoustic transducers is configured to record at least one of airborne sound and structure-borne sound.

25. The device as claimed in claim 13, wherein device for checking the medical device in incorporated in said medical device, said medical device further comprising a vibration device.

26. The device as claimed in claim 13, wherein at least one of said one or more acoustic transducers is situated near a motor of said medical device for detecting sound emitted by said motor.

27. The device as claimed in claim 26, wherein said at least one acoustic transducer is situated near a moving part and coupled to said motor of said medical device for detecting sound emitted by said moving part.

28. The device as claimed in claim 13, wherein at least one of said one or more acoustic transducers is situated on a housing of said medical device for detecting sounds emitted by said housing.

29. An analysis station for a medical device comprising:
a communications device for communicatively coupling said medical device to said analysis station;
a device for checking said medical device, said device for checking comprising one or more acoustic transducers configured to emit and receive signals;
a recording device coupled to said device for checking and configured to record data received from said device for checking; and
an evaluation unit coupled to said recording device for processing characteristics related to said medical device.

30. The analysis station as claimed in claim 29, wherein said one or more acoustic transducers are configured to receive at least one of structure-borne sound and airborne sound from said medical device or said device for checking.

31. The diagnosis station as claimed in claim 29, wherein said recording device is configured to record data received from an acoustic transducer coupled to, disposed on, or integrated with the medical device.

32. A non-implantable medical device comprising:
one or more acoustic transducers, wherein at least one of said one or more acoustic transducers detects sound characteristics related to at least one of the structure or performance of said medical device; and
a communication device for communicating said detected sound characteristics with a device for checking said medical device.

33. A method for checking a medical device comprising:
providing acoustic transducer signals;
detecting the acoustic transducer signals;
analyzing said transducer signals; and
categorizing the status of said medical device based on said analyzed transducer signals, said status of said medical device indicative that the medical device is functioning correctly, that the medical device functioning is deviating from a prescribed pattern, or that a fault associated with the medical device has occurred.

34. The method of claim 33, wherein categorizing the status of said medical device comprises signalling said status via at least one of a visual or an audible signal.

35. The method of claim 33, wherein detecting said acoustic transducer signals comprises at least one of continuously or temporarily detecting said acoustic transducer signals.

36. The method of claim 33, further comprising detecting an impact on the medical device based on the analysis of the transducer signals.

37. The method of claim 33, further comprising at least one of outputting a warning signal or blocking the medical device based on a detected fault the detected functioning of the medical device, said fault or functioning processed by means for analyzing the detected sound.

38. The method of claim 33, further comprising storing the transducer signals detected or the result of the analysis of the transducer signals.

39. The method of claim 33, wherein said acoustic transducer signals are the result of an oscillation, vibration, or impact produced in or on said medical device.
